(19) United States
(12) Patent Application Publication
(10) Pub. No.: US 2022/0044778 A1
(43) Pub. Date: Feb. 10, 2022

(54) METHOD AND ELECTRONIC APPARATUS FOR PROVIDING CLASSIFICATION DATA OF ELECTROCARDIOGRAM SIGNALS

(71) Applicant: ATSENS CO., LTD., Gyeonggi-do (KR)

(72) Inventors: Jong Ook JEONG, Gyeonggi-do (KR); Chang Ho LEE, Gyeonggi-do (KR)

(73) Assignee: ATSENS CO., LTD., Gyeonggi-do (KR)

(21) Appl. No.: 17/350,618

(22) Filed: Jun. 17, 2021

(30) Foreign Application Priority Data
Aug. 6, 2020 (KR) 10-2020-0098824

(51) Int. Cl. G16H 15/00; A61B 5/349; G16H 50/20; G16H 40/67; G01R 23/16

(52) U.S. Cl. CPC G16H 15/00; A61B 5/349; A61B 5/339; G16H 40/67; G01R 23/16; G16H 50/20

(57) ABSTRACT

A method for providing classification data of an electrocardiogram signal includes loading, by an electronic apparatus, an electrocardiogram signal, analyzing, by the electronic apparatus, the electrocardiogram signal and generating labels related to the electrocardiogram signal in association with the electrocardiogram signal, determining, by the electronic apparatus, category values of signal segments of the electrocardiogram signal based on labels related to the electrocardiogram signal according to standard classification criteria, displaying, by the electronic apparatus, labels related to the electrocardiogram signal and category values of segments of the electrocardiogram signal on an output unit, in response to a selection input for a first category value from among the category values, generating, by the electronic apparatus, information regarding an electrocardiogram signal related to the first category value, and displaying information regarding the electrocardiogram signal related to the first category value on the output unit.

### CROSS-REFERENCE TO RELATED APPLICATION

This application is based on and claims priority under 35 U.S.C. § 119 to Korean Patent Application No. 10-2020-0098824, filed on Aug. 6, 2020, in the Korean Intellectual Property Office, the disclosure of which is incorporated by reference herein in its entirety.

### BACKGROUND

### 1. Field

One or more embodiments relate to a method and an electronic apparatus for providing classification data of electrocardiogram signals.

### 2. Description of the Related Art

As a portable electrocardiogram measuring device known to date, a Holter electrocardiometer is used by receiving a first prescription from a hospital, attaching the first prescription for 24 hours, and then being admitted to the hospital again or returning the Holter electrocardiometer by visiting the hospital again. Electrocardiogram signals sensed for 24 hours are stored in an internal memory of an electrocardiogram measuring device like a Holter electrocardiometer, and the stored electrocardiogram signals are downloaded to a PC of an analyst. The analyst may analyze downloaded electrocardiogram data using analysis software and generate an analysis report including analysis statistics and summary information. An analysis report generated by the analyst is provided to a medical specialist, and the medical specialist reviews the analysis report and derives a diagnosis result based on a result of the review.

As a portable electrocardiogram measuring device, the Holter electrocardiometer is designed for a short-term measurement and arrhythmia diagnosis from among heart diseases. Recently, patch-type electrocardiogram measuring devices that replace the existing Holter electrocardiometer are being released. Unlike the conventional Holter electrocardiometer, patch-type electrocardiogram measuring devices exhibit good wearability, small sizes, water-resistance, and long-term measurement.

Measuring electrocardiogram signal data over a long period of time may significantly increase the probability of diagnosing heart disease. For example, it is reported that the arrhythmia diagnosis rate is about 30% when electrocardiogram signal data is measured for one day, the arrhythmia diagnosis rate is more than 90% when electrocardiogram signal data is measured for 7 days or more, and the arrhythmia diagnosis rate is nearly 100% when electrocardiogram signal data is measured for 14 days.

However, when measured for a long time, an amount of electrocardiogram signal data stored increases proportionally, and thus, it takes a significant amount of time for analysts to analyze data and derive an analysis report. For example, in the case of patients with severe arrhythmia, it takes more than 2 hours to analyze data stored by a conventional Holter electrocardiometer for 24 hours. Therefore, a significant analysis time may be expected to analyze data stored for a long period of time of 7 days or longer, which is practically difficult to cope with by a limited number of analysts.

This is a significant impeding factor in the conventional analysis method despite of high diagnostic performance based on long-term use. Therefore, in the case of storing electrocardiogram signal data for a long period of time, a method that enables rapid analysis while improving diagnostic performance is demanded.

### SUMMARY

One or more embodiments provide a method and an electronic apparatus for providing classification data of an electrocardiogram signal by generating a label that classifies an electrocardiogram signal according to category values and providing signal waveforms according to the category values, thereby reducing the time for analyzing an electrocardiogram signal.

Additional aspects will be set forth in part in the description which follows and, in part, will be apparent from the description, or may be learned by practice of the presented embodiments of the disclosure.

According to one or more embodiments, a method for providing classification data of an electrocardiogram signal, the method includes loading, by an electronic apparatus, an electrocardiogram signal; analyzing, by the electronic apparatus, the electrocardiogram signal and generating labels related to the electrocardiogram signal in association with the electrocardiogram signal; determining, by the electronic apparatus, category values of signal segments of the electrocardiogram signal based on labels related to the electrocardiogram signal according to standard classification criteria; displaying, by the electronic apparatus, labels related to the electrocardiogram signal and category values of segments of the electrocardiogram signal on an output unit; in response to a selection input for a first category value from among the category values, generating, by the electronic apparatus, information regarding an electrocardiogram signal related to the first category value; and displaying information regarding the electrocardiogram signal related to the first category value on the output unit.

In at least one variant, the information regarding the electrocardiogram signal related to the first category value may include information regarding the number of signal segments of the first category value or a representative signal waveform of the first category value.

In another variant, the information regarding the representative signal waveform of the first category value may include the number of representative signal waveforms or the number of signal segments of each representative signal waveform.

In further another variant, the method may further include displaying signal waveforms of signal segments classified based on representative signal waveforms of the first category value on the output unit in response to a selection input for information regarding the representative signal waveform of the first category value.

In another variant, the method may further include, in response to a correction input for a first signal segment of the first category value, correcting, by the electronic apparatus, classification data to set a category value of the first signal segment to a second category value and to classify the first signal segment by a label including the second category value.

In another variant, the method may further include, generating, by the electronic apparatus, a report file by converting information regarding the category values of the electrocardiogram signal and representative signal waveforms of the category values into a report format.

In another variant, labels may be generated in association with the signal by classifying the electrocardiogram signal according to pre-set time intervals and generate labels for respective signal intervals of the time intervals.

Labels may be generated in association with the signal by classifying the electrocardiogram signal according to pre-set heart rates and generate labels for respective signal intervals of the heart rates.

In another variant, an electronic apparatus may load an electrocardiogram signal by receiving an electrocardiogram signal measured in real time from an electrocardiogram measuring device connected through a communicator or receiving an electrocardiogram signal stored in an external device connected through the communicator.

In another variant, a category value is a sub-concept of a label, and one label may be defined by including one or more category values.

Information regarding an electrocardiogram signal related to a first category value is displayed on an output unit by displaying information including the number of representative signal waveforms of the first category value and the number of signal segments of the first category value and then, based on an additional input, displaying information including the number of signal segments respectively of representative signal waveforms of the first category value.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1A is a configuration diagram of an electronic apparatus displaying a result of analyzing a bio-signal according to one or more embodiments.

FIG. 1B is a configuration diagram of another electronic apparatus displaying a result of analyzing a bio-signal according to one or more embodiments.

FIG. 2 is a diagram showing an example of classification of category values by the electronic apparatus 100 according to one or more embodiments.

FIG. 3 is a diagram showing an example of bio-signals and classification data output according to one or more embodiments.

FIG. 4 is a diagram showing an example of information corresponding to category values.

FIG. 5 is an exemplary diagram of signal segments including a missing signal peak.

FIG. 6 is an exemplary diagram of a signal segment in which one signal peak is missed compared to a normal waveform.

FIG. 7 is an exemplary diagram of a signal segment in which one signal peak is missed compared to a normal waveform.

FIG. 8 is a diagram showing example of first user interface, and FIG. 9 is a diagram showing example of second user interface provided according to one or more embodiments.

FIG. 10 is a flowchart of a method for processing a label corresponding to an electrocardiogram signal according to one or more embodiments.

FIG. 11 is a flowchart of a method of further acquiring and processing an electrocardiogram signal and a bio-signal other than the electrocardiogram signal according to one or more embodiments.

FIG. 12A is a diagram for a normal signal waveform.

FIG. 12B is a diagram for a signal waveform corresponding to Atrial Fibrillation.

FIG. 12C is an exemplary diagram of adding other information to a signal segment.

FIG. 13 is a flowchart of a method of providing classification data of an electrocardiogram signal according to one or more embodiments.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Hereinafter, the configuration and operation of one or more embodiments will be described in detail with reference to embodiments of one or more embodiments shown in the accompanying drawings.

One or more embodiments may include various embodiments and modifications, and embodiments thereof will be illustrated in the drawings and will be described herein in detail. The effects and features of one or more embodiments and the accompanying methods thereof will become apparent from the following description of the embodiments, taken in conjunction with the accompanying drawings. However, one or more embodiments are not limited to the embodiments described below, and may be embodied in various modes.

Reference will now be made in detail to embodiments, examples of which are illustrated in the accompanying drawings. In the drawings, the same elements are denoted by the same reference numerals, and a repeated explanation thereof will not be given.

In this specification, terms such as “learning” are not intended to refer to human mental processes such as educational activities, but should be interpreted as terms referring to performing machine learning through computing according to procedures.

It will be understood that although the terms “first”, “second”, etc. may be used herein to describe various elements, these elements should not be limited by these terms. These elements are only used to distinguish one element from another.

As used herein, the singular forms “a,” “an” and “the” are intended to include the plural forms as well, unless the context clearly indicates otherwise.

It will be further understood that the terms “comprises” and/or “comprising” used herein specify the presence of stated features or components, but do not preclude the presence or addition of one or more other features or components.

Sizes of elements in the drawings may be exaggerated for convenience of explanation. In other words, since sizes and thicknesses of components in the drawings are arbitrarily illustrated for convenience of explanation, the following embodiments are not limited thereto.

When a certain embodiment may be implemented differently, a specific process order may be performed differently from the described order. For example, two consecutively described processes may be performed substantially at the same time or performed in an order opposite to the described order.

In the present specification, a bio-signal data stream is obtained by converting bio-signals into electronic data and may include values of bio-signals.

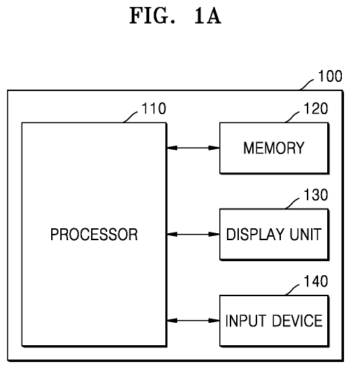

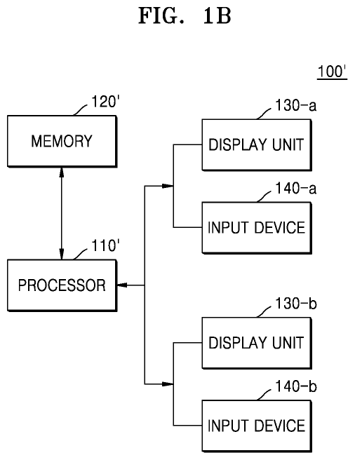

FIGS. 1A and 1B are configuration diagrams of an electronic apparatus 100 displaying a result of analyzing a bio-signal according to one or more embodiments. As shown in FIG. 1A, the electronic apparatus 100 according to one or more embodiments may include a processor 110, a memory 120, a display 130, and an input device 140.

The processor 110 may classify a bio-signal data stream based on one or more category values and display signal waveforms classified based on the category values on the display 130. The bio-signal data stream refers to original data of bio-signals measured for a set period of time. The bio-signal data stream may be measured for 1 week or 1 day, for example. The bio-signal data stream may be electrocardiogram signals, and a data stream measured for 1 week may have a size of several hundreds of megabytes.

The processor 110 may divide a measured bio-signal data stream into one or more signal segments. Signal segments may correspond to category values according to predetermined classification criteria. Category values are stored in the memory 120 in association with the bio-signal data stream.

In another embodiment, the processor 110 may display signal waveforms of a bio-signal data stream in a time series, where the processor 110 may generate classified category value as labels and display the labels on the display 130 in correspondence to the signal waveforms. Also, the processor 110 may display signal waveforms of a category value selected by a user input in a time series or may display at least one representative signal waveform of the selected category value.

The bio-signal data stream may be stored in the memory 120. The input device 140 obtains a user's input and may include a keyboard, a mouse, a microphone, a joystick, etc. Although FIG. 1A shows that the electronic apparatus 100 is a standalone system, one or more embodiments are not limited thereto, and the electronic apparatus 100 may be implemented as a cloud computing device. As shown in FIG. 1B, an electronic apparatus 100′ implemented as a cloud computing device may include a remote memory 120′, a remote display 130-a, and a remote input device 140-a. A processor 110′ may receive a bio-signal data stream such as electrocardiogram data stored in the remote memory unit 120′. The processor 110′ of the electronic apparatus 100′ may receive a bio-signal data stream from the remote memory unit 120′ and output classification data of the bio-signal data stream through the remote display 130-a.

Also, the electronic apparatus 100′ may include a plurality of displays 130-a and 130-b and a plurality of input devices 140-a and 140-b. One display 130-a and one input device 140-a may be used by a first analyst and the other display 130-b and the other input device 140-b may be used by a second analyst. Therefore, classification of bio-signals may be performed with the cooperation of the first analyst and the second analyst. Although FIG. 1B shows two displays and input devices, the one or more embodiments are not limited thereto, and the electronic apparatus 100′ may include one more displays and one more input devices.

Here, the bio-signal data stream may be an electrocardiogram signal data stream, for example. In other words, an electrocardiogram signal data stream measured over a long period of time may be classified by the processor 110 into a plurality of signal segments according to category values. The electrocardiogram signal data stream may be segmented into signal segments by using a particular peak value, e.g., an R-peak value. Also, the electrocardiogram signal data stream may be segmented into predetermined periods.

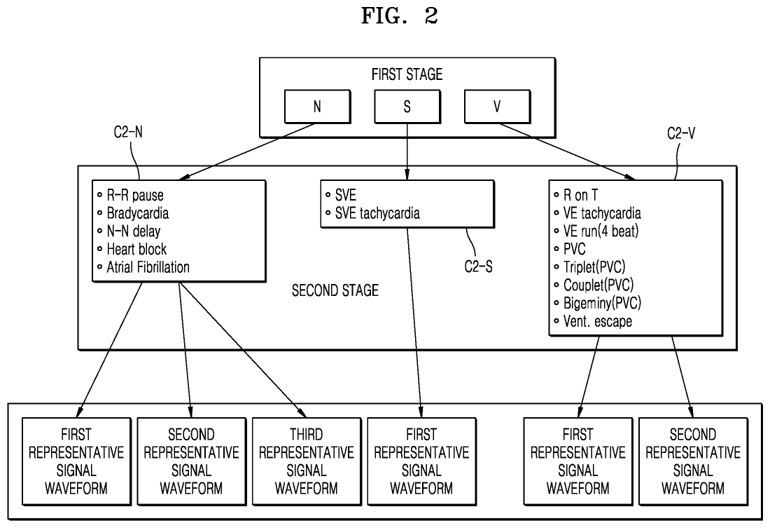

FIG. 2 is a diagram showing an example of classification of category values by the electronic apparatus 100 according to one or more embodiments.

As shown in FIG. 2, in a first stage, an electrocardiogram signal data stream may be classified into category values including a normal beat (N), a bundle branch block, supraventricular ectopy beat (SVEB; S), and a ventricular ectopy beat (VEB; V) according to predetermined classification criteria. Here, the classification criteria may include IEC 6061-2-47, which is an international standard, or CIEC 6061-2-47, which is a domestic standard, but are not limited thereto. Also, the classification criteria may be changed by a user.

In the first stage, N, S, and V may be classified into category values of a second stage, respectively. The category values of N may be reclassified into category values of C2-N. The category values of S may be reclassified into category values of C2-S. The category values of V may be reclassified into category values of C2-V.

According to one or more embodiments, an electronic device 100 or 100′ may classify the electrocardiogram signal data stream based on category values C2-N, C2-S, and C2-V. The electronic device 100 or 100′ may classify the electrocardiogram signal data stream according to representative signal waveforms of respective categories. For the electrocardiogram signal data stream, signal segments may be classified according to category values and representative signal waveforms of the category values. Here the signal segment may be all or part of the electrocardiogram signal. The signal segment may be for dividing the electrocardiogram signal by a predetermined length.

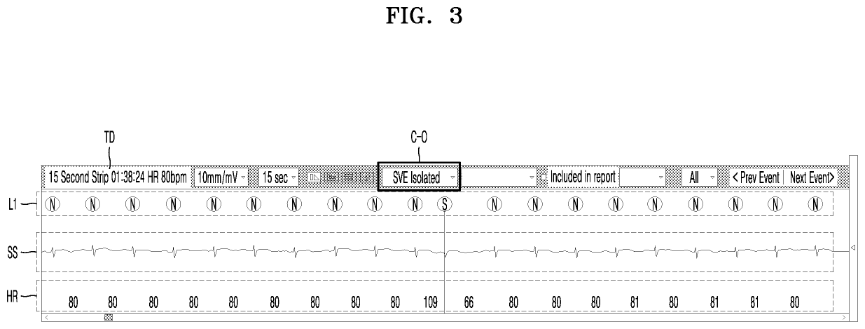

As shown in FIG. 3, the category values of N, S, and V in the first stage may be transformed to labels and included in the electrocardiogram signal data stream. Labels including category values of N, S, and V may be displayed while the electrocardiogram signal data stream is displayed. When the resolution at which the electrocardiogram signal data stream is displayed is adjusted, a label including the category values of the second step may be output through the display unit. The label may be generated corresponding to the signal segment of the electrocardiogram signal data stream. The electrocardiogram signal data stream may be divided into one or more signal segments.

When a selection input for the one or more signal segments labeled as “S” is received, intervals of time information of the one or more signal segments may be adjusted based on an input time of the selection input and the one or more signal segments may be output. For example, the interval of time information of the one or more signal segments may increase from 1 second to 5 seconds or may decrease from 1 second to 0.1 seconds. When an interval of time information of the one or more signal segments increases, the one or more signal segments are displayed in a reduced size. When an interval of time information of the one or more signal segments decreases, the one or more signal segments may be displayed in an enlarged size.

The one or more signal segments may be output while including a label of the first stage and/or a label of the second stage. A user may change label information regarding the one or more signal segments through a selection input for a label. Because the label information regarding the one or more signal segments is related to category values, when a label of first signal segment is modified, a category value of the first signal segment may also be modified.

An electrocardiogram signal data stream may include a label display region L1, a signal display region SS, a heart rate display region HR, a category option selecting region C-O, and a time interval information display region TD.

In the label display region L1, labels corresponding to signal segments of the electrocardiogram signal data stream may be displayed. Labels may correspond to category values, but one or more embodiments are not limited thereto.

The electrocardiogram signal data stream may be displayed in the signal display region SS.

Heart rate information of the electrocardiogram signal data stream may be displayed in the heart rate display region HR. In the category option selecting region C-0, levels of labels to be displayed may be selected.

A labeled electrocardiogram signal shown in FIG. 3 is generated by processing the first stage and the second stage through an analysis algorithm according to one or more embodiments. An analyst may check output data of FIG. 3 through an output device and may scan electrocardiogram signal data and modify one or more label of an electrocardiogram signal or perform special marking. A result thereof may be generated as a report and transmitted to a medical specialist. After checking the report, the medical specialist may request additional analysis of the electrocardiogram signal from the analyst.

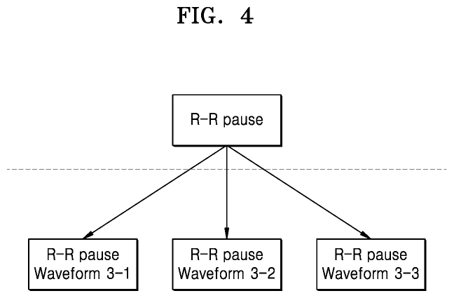

FIG. 4 is a diagram showing an example of information corresponding to category values.

According to embodiments of one or more embodiments, labels of a signal data stream may be generated in correspondence to category values of the first stage or the second stage. Also, a signal data stream may be classified according to representative signal waveforms of R-R pause, e.g., a waveform 3-1, a waveform 3-2, and a waveform 3-3, according to 3-stage classification criteria. According to the 3-stage classification criteria, the electronic apparatus 100 or 100′ may categorize and provide signal segments of the category value of ‘R-R pause’ according to representative signal waveforms 3-1, 3-2, and 3-3.

According to one or more embodiments, representative signal waveforms of category values and information regarding the representative signal waveforms may be provided.

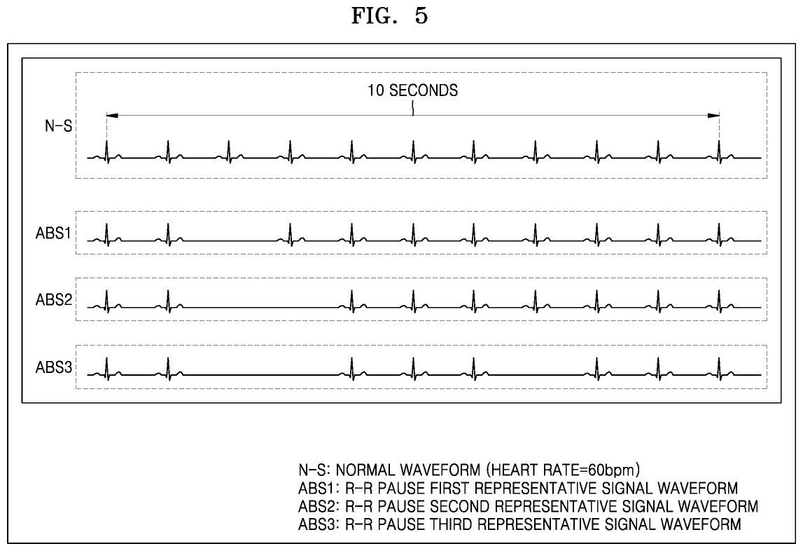

A first representative signal waveform 3-1 may be the same as ABS1 of FIG. 5 and may be a signal waveform without one R peak as compared to a normal waveform N-S. A second representative signal waveform 3-2 may be the same as ABS2 of FIG. 5 and may be a signal waveform without two R peaks as compared to the normal waveform N-S. A third representative signal waveform 3-3 may be the same as ABS3 of FIG. 5 and may be a signal waveform without three R peaks as compared to the normal waveform N-S.

First to third representative signal waveforms of FIG. 5 are merely examples, and one or more embodiments are not limited thereto. By analyzing an electrocardiogram signal data stream, representative signal waveforms may be determined. As shown in FIG. 5, the first to third representative signal waveforms may be determined as having a duration of 10 seconds. Representative signal waveforms may be arranged according to frequencies. For example, representative signal waveforms may be arranged in the order of high frequency. However, one or more embodiments are not limited thereto, and the order of the arrangement of representative signal waveforms may be determined considering an object's past medical history, or based on priorities of the representative signal waveforms.

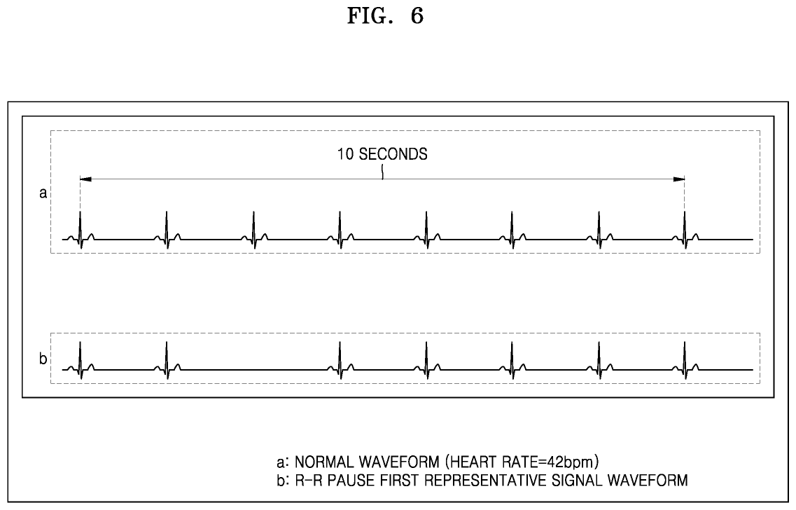

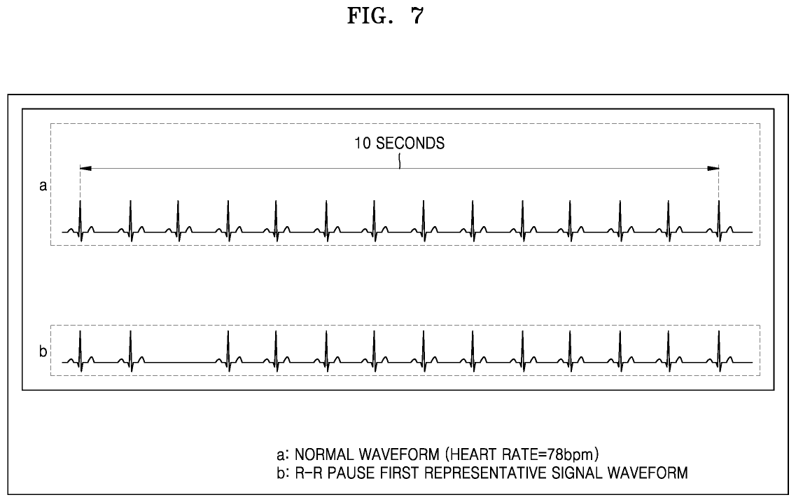

Representative signal waveforms may be determined as shown in FIGS. 6 and 7.

The electronic apparatus 100 or 100′ may determine representative signal waveforms considering the size of a signal interval. For example, as shown in FIG. 7, the electronic apparatus 100 or 100′ may obtain a representative signal waveform by analyzing a set time interval in a situation of a first heart rate. For example, a representative signal waveform in which one signal peak from among eight peak values is omitted may be extracted. The electronic apparatus 100 or 100′ may obtain a representative signal waveform by analyzing a set time interval in the situation of a second heart rate. For example, a representative signal waveform in which two signal peaks from among fourteen peak values are omitted may be extracted. As shown in FIGS. 6 and 7, the set time interval may be 10 seconds.

As described above, it is obvious that the electronic apparatus 100 or 100′ may obtain a representative signal waveform in various heart rate situations. Because a representative signal waveform may be generated only in a situation of a particular heart rate, the representative signal waveform may provide information regarding a heart rate of a corresponding signal together.

As described above, the electronic apparatus 100 or 100′ may divide a signal data stream into one or more signal segments based on time, but may also divide a signal data stream into one or more signal segments based on heart rates.

The electronic apparatus 100 or 100′ may calculate missing signal peaks in a section including 10 signal peaks in a signal data stream or a signal segment. Calculation of missing signal peaks may be automatically processed through signal normalization in the time domain.

Also, in the electronic apparatus 100 or 100′, when an impedance value between a measuring electrode and the skin of an object is changed, the magnitude of a signal may change. In this case, by normalizing the signal data stream to a set magnitude value, representative signal waveforms in the signal data stream may be determined. For example, the set magnitude value may be an average value of particular signal peak values included in the signal data stream, e.g., an average of R-peak values.

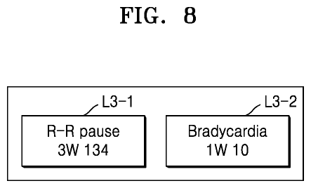

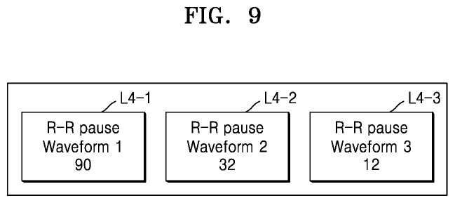

FIG. 8 is a diagram showing example of first user interface, and FIG. 9 is a diagram showing example of second user interface provided according to one or more embodiments.

The electronic apparatus 100 or 100′ may generate information corresponding to an R-R pause and a Bradycardia, which are category values of classification criteria. As shown in FIG. 8, information L3-1 corresponding to a category value may include the number 134 of signal segments having a category value of RR pause and the number 3W of representative signal waveforms of the category value of RR pause.

The electronic apparatus 100 or 100′ may provide the numbers 90, 32, and 12 of signal segments classified as representative signal waveforms of an R-R pause, which is a category value of classification criteria.

After the electronic apparatus 100 or 100′ may provide the number 134 of signal segments having a category value as shown in FIG. 9 and the number 3W of representative signal waveforms of a category value, the electronic apparatus 100 or 100′ may provide the numbers 90, 32, and 12 of signal segments classified as representative signal waveforms of a category value as information L4-1, L4-2, and L4-3. Accordingly, information L4-2 regarding the representative signal waveform that is most frequently generated from among representative signal waveforms of the category value may be easily checked.

When an analyst selects Information or label for representative signal waveform, waveforms of signal segments corresponding to the representative signal waveform are listed (not shown). Because the occurrence frequency of a R-R pause waveform 1 L4-1 of FIG. 9 is ninety (90), 90 signal waveforms may be displayed on a screen. Ninety (90) signal waveforms corresponding to R-R pause waveform 1 may have a predetermined length of time. An analyst may determine a corresponding signal waveform by using an adjacent signal waveform and correct a wrong label of the corresponding signal waveform. In critical situations, a label of the corresponding signal waveform may be added. Therefore, the analyst may search for an erroneous label considering the occurring frequencies of signal waveforms. The analyst may select and check only signal waveforms with high occurring frequencies or signal waveforms with low occurring frequencies.

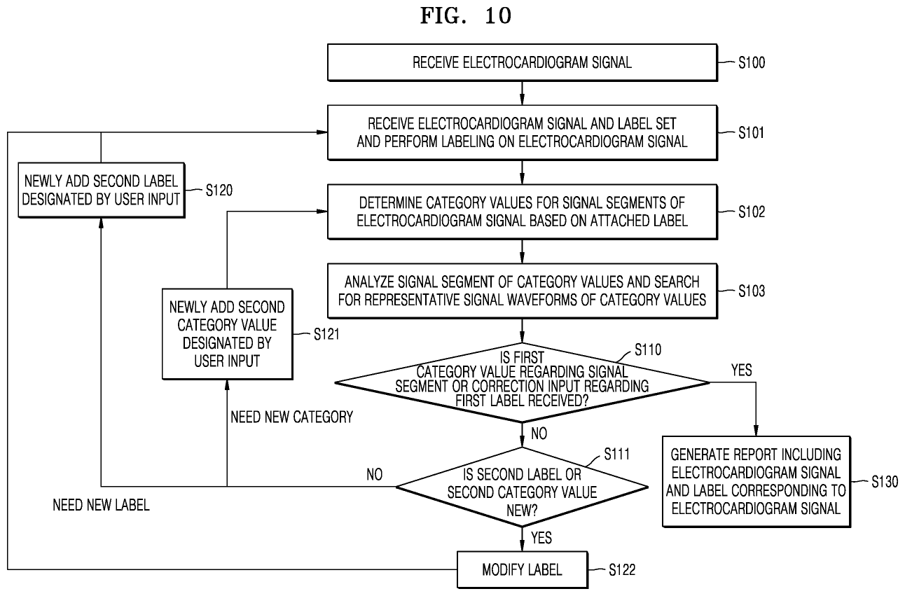

FIG. 10 is a flowchart of a method for processing a label corresponding to an electrocardiogram signal according to one or more embodiments.

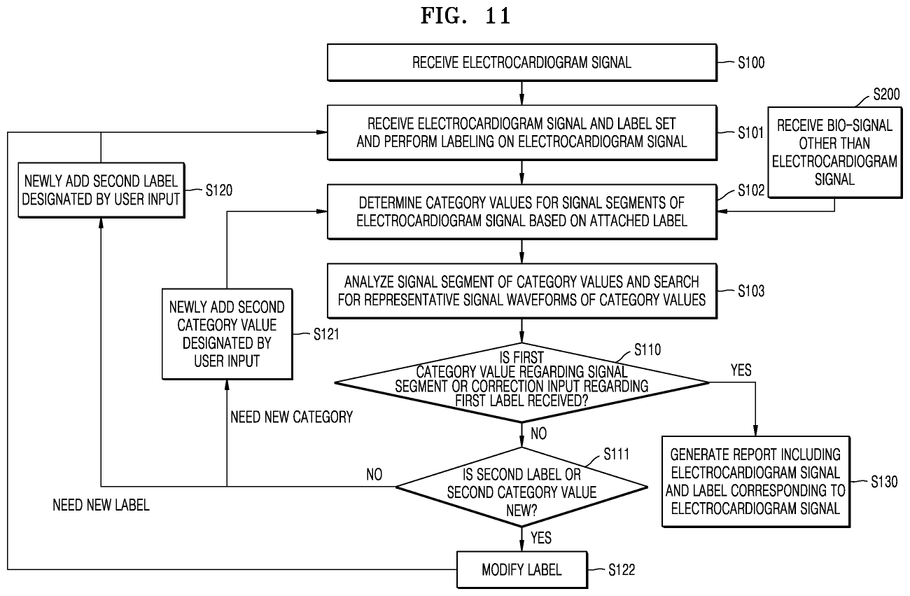

FIG. 11 is a flowchart of a method of further acquiring and processing an electrocardiogram signal and a bio-signal other than the electrocardiogram signal according to one or more embodiments.

In operation S100, the electronic apparatus 100 or 100′ receives an electrocardiogram signal.

In operation S101, the electronic apparatus 100 or 100′ receives an electrocardiogram signal and a labels set and performs a labeling process on the electrocardiogram signal. The electronic apparatus 100 or 100′ performs a labeling process according to the classification criteria of the first stage and the second stage of FIG. 2. Labels corresponding to the classification criteria are inserted to signal segments of the electrocardiogram signal. In S101, a labeling process may be performed using a conventional labeling algorithm. Recently, machine learning or neural networks are being used. However, for the level required in medical applications, a process of final confirmation by a medical specialist is being performed.

In operation S102, the electronic apparatus 100 or 100′ determines a category value of the signal segments of the electrocardiogram signal based on attached labels. Here, the category value may be one of the 3-stage category values as defined in FIG. 2.

The electronic apparatus 100 or 100′ may search for representative signal waveforms of the category value by analyzing signal segments of the category value (operation S103). One category value may be classified into one or more representative signal waveforms. From among electrocardiogram signals, signal waveforms of the corresponding category value may be selected, and representative signal waveforms may be extracted considering the occurrence frequency from among the signal waveforms. Information regarding the category value and the representative signal waveforms of the category value may be displayed as shown in FIGS. 8 and 9.

The electronic apparatus 100 or 100′ may receive a confirmation input for a category value or a label for a signal segment. The electronic apparatus 100 or 100′ checks whether a correction input for a first category value is received (operation S110). The electronic apparatus 100 or 100′ may correct the first category value for the signal segment to a second category value in response to the correction input for the first category value.

At this time, when the second category value is a new category value, the electronic apparatus 100 or 100′ may newly add the second category value designated by a user input (operation S121).

The electronic apparatus 100 or 100′ may receive a correction input for a first label. The electronic apparatus 100 or 100′ may correct a first label for the signal segment to a second label in response to the correction input for the first label.

When the second label is a new label, the electronic apparatus 100 or 100′ may add the second label in correspondence to an electrocardiogram signal (operation S120).

The electronic apparatus 100 or 100′ may add a newly added label or category value to the classification criteria. The classification criteria shown in FIG. 2 may be modified.

The electronic apparatus 100 or 100′ may generate a report including an electrocardiogram signal and a label corresponding thereto (operation S130).

In another embodiment, when providing classification data generated by classifying electrocardiogram signals of objects having a particular heart disease, a corresponding particular heart disease and a first representative signal waveform having the highest occurrence frequency may be matched to each other. When the occurrence frequency of a second representative signal waveform other than the first representative signal waveform is equal to or greater than a pre-set threshold value, the electronic apparatus 100 or 100′ may newly add a category value corresponding to the second representative signal waveform.

In another embodiment, when electrocardiogram signals of a patient are received from a measuring device, the electronic apparatus 100 or 100′ may additionally perform a process of classifying electrocardiogram signals based on a label and a category value.

The process of classifying electrocardiogram signals based on a label or a category value may be performed by a learning algorithm generated through machine learning or neural networks. The learning algorithm may be trained based on labels or category values input by a user.

Operation S110 or operation S111 may be performed based on an input by a user or may be performed by artificial intelligence. In other words, an input may be generated by a computing device of artificial intelligence.

As described above, a label or a category value corresponding to a representative signal waveform that is not classified as a conventional representative signal waveform may be added to the classification criteria of FIG. 2. After the third stage (refer to FIG. 8) is executed, the electronic apparatus 100 or 100′ may sequentially execute a fourth stage (refer to FIG. 9). In the third stage, the signal segments can be classified into one or more categories. The signal segments classified can be further classified into the next level category.

According to one or more embodiments, information regarding a representative signal waveform having a high occurrence frequency may be easily checked by using information regarding the occurrence frequency for each representative signal waveform of a category provided by the electronic apparatus 100 or 100′, and a user may more intensively check representative signal waveforms with high occurrence frequencies. Therefore, checking an entire electrocardiogram signal may become easier, and analysis errors may be reduced.

The electronic apparatus 100 or 100′ may display representative signal waveforms of a category in response to a selection input from a user. For example, normalized signal waveforms may be overlapped and displayed or non-normalized waveforms may be additionally divided and displayed.

As shown in FIG. 11, when generating a category of an electrocardiogram signal, other bio-signals may be used.

The electronic apparatus 100 or 100′ may obtain bio-signals (motion, respiration, blood pressure, oxygen saturation, etc.) other than an electrocardiogram signal (operation S200). For example, in relation to a signal segment having a category value of an RR pause according to the classification criteria of an electrocardiogram signal, when a momentum is more than a pre-set reference value, a category value that combines RR pause with a high momentum may be assigned in correspondence to the signal segment. Because the heart is the center of human biological signals, the electronic apparatus 100 or 100′ may analyze other biological signals based on the electrocardiogram signal (or ECG data).

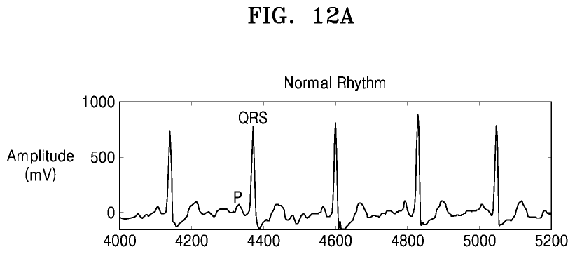

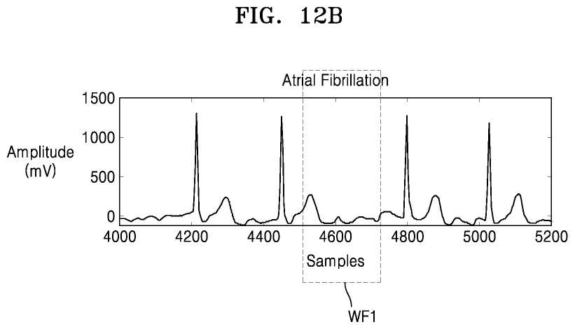

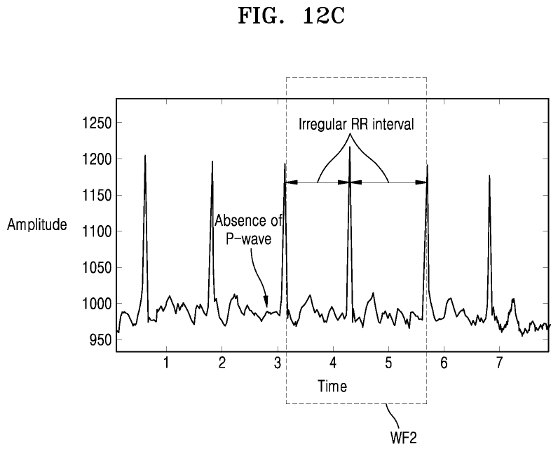

FIG. 12A is a diagram for a normal signal waveform, and FIG. 12B is a diagram for a signal waveform corresponding to Atrial Fibrillation.

FIG. 12C is an exemplary diagram of adding other information to a signal segment. According to a predetermined classification criteria, a signal segment is set to one of an RR pause category, a bradycardia category, and an atrial fibrillation category. As shown in FIG. 12B, for a signal waveform WF1 in which a P-peak missing and an RR pause occur simultaneously, a P-peak missing and an RR pause may be newly added to categories. As shown in FIG. 12C, for a waveform WF2, a P-peak missing and an arrhythmia may be newly added to categories by user input or user instruction.

This is because causes of heart problems appear as a variety of results. Therefore, through accumulations as stated above, an analyst or artificial intelligence may make an accurate judgment. Additional analysis may be added to an algorithm, and a new category may be added, additional category information may be added, or a comment may be added.

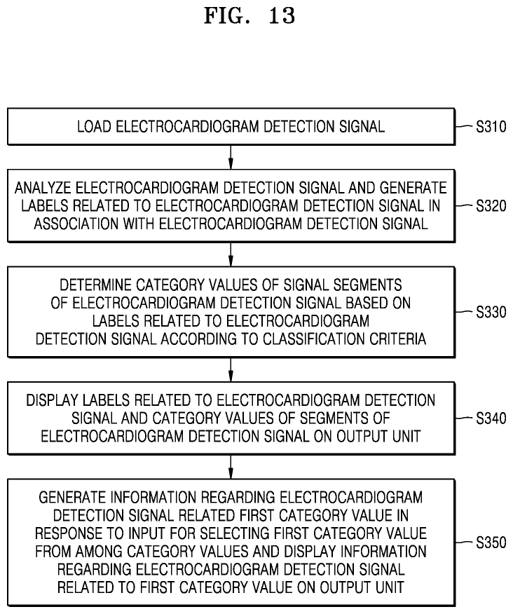

FIG. 13 is a flowchart of a method of providing classification data of an electrocardiogram signal according to one or more embodiments.

In operation S310, an electronic apparatus may load an electrocardiogram signal.

In operation S320, the electronic apparatus analyzes the electrocardiogram signal and generates labels related to the electrocardiogram signal in association with the electrocardiogram signal.

In operation S330, the electronic apparatus determines category values of signal segments of the electrocardiogram signal based on labels related to the electrocardiogram signal according to standard classification criteria.

In operation S340, the electronic apparatus displays labels related to the electrocardiogram signal and category values of segments of the electrocardiogram signal through an output unit.

In operation S350, the electronic apparatus generates information regarding the electrocardiogram signal related to a first category value in response to a selection input for the first category value from among the category values and displays information regarding the electrocardiogram signal related to the first category value on the output unit.

The information regarding the electrocardiogram signal related to the first category value may include information regarding the number of signal segments of the first category value or a representative signal waveform of the first category value.

The information regarding the representative signal waveform of the first category value may include the number of representative signal waveforms or the number of signal segments of each representative signal waveform.

The electronic apparatus may display signal waveforms of signal segments classified based on representative signal waveforms of the first category value on the output unit in response to a selection input for information regarding the representative signal waveform of the first category value.

In response to a correction input for a first signal segment of the first category value, the electronic apparatus may correct classification data to set a category value of the first signal segment to a second category value, and to classify the first signal segment by a label including the second category value.

The electronic apparatus may generate a report file by converting information regarding the category values of the electrocardiogram signal and representative signal waveforms of the category values into a report format.

Labels may be generated in association with the signal by classifying the electrocardiogram signal according to pre-set time intervals and generating labels for respective signal intervals of the time intervals. Labels may be generated in association with the signal by classifying the electrocardiogram signal according to pre-set heart rates and generating labels for respective signal intervals of the heart rates.

An electronic apparatus may load an electrocardiogram signal by receiving an electrocardiogram signal measured in real time from an electrocardiogram measuring device connected through a communicator or receiving an electrocardiogram signal stored in an external device connected through the communicator.

A category value is a sub-concept of a label, and one label may be defined by including one or more category values.

Information regarding an electrocardiogram signal related to a first category value is displayed on an output unit by displaying information including the number of representative signal waveforms of the first category value and the number of signal segments of the first category value and then, based on an additional input, displaying information including the number of signal segments respectively of representative signal waveforms of the first category value.

The apparatus described above may be implemented as a hardware component, a software component, and/or a combination of hardware components and software components. For example, the devices and components described in the embodiments may be implemented using one or more general purpose or special purpose computers, e.g., a processor, a controller, an arithmetic logic unit (ALU), a digital signal processor, a microcomputer, a field programmable gate array (FPGA), a programmable logic unit (PLU), a microprocessor, or any other device capable of executing and responding to instructions. A processing device may execute an operating system (OS) and one or more software applications running on the OS. The processing device may also access, store, manipulate, process, and generate data in response to execution of software. For the convenience of explanation, it has been described above that one processing device is used. However, it would be obvious to one of ordinary skill in the art that the processing device may include a plurality of processing elements and/or a plurality of types of processing elements. For example, the processing device may include a plurality of processors or one processor and one controller. Also, other processing configurations like parallel processors may be employed.

The software may include a computer program, code, instructions, or a combination of one or more of the foregoing, to configure the processing device to operate as demanded or to command the processing device independently or collectively. For the purpose of interpreting or providing instructions or data to the processing device, software and/or data may be permanently or temporarily embodied in any type of machine, component, physical device, virtual equipment, computer storage medium, or a signal wave to be transmitted. The software may be distributed over networked computer systems so that it may be stored or executed in a distributed manner. The software and data may be stored on one or more computer-readable recording media.

The methods according to embodiments may be embodied in the form of program instructions that may be executed by various computer means and recorded on a computer-readable recording medium. The computer-readable recording media may include program instructions, data files, and data structures alone or a combination thereof. The program commands recorded on the medium may be specially designed and configured for example embodiments or may be published and available to one of ordinary skill in computer software. Examples of the computer-readable recording medium include a hardware device specially configured to store and perform program instructions, for example, a magnetic medium, such as a hard disk, a floppy disk, and a magnetic tape, an optical recording medium, such as a CD-ROM, a DVD, and the like, a magneto-optical medium, such as a floptical disc, ROM, RAM, a flash memory, and the like. Examples of program commands include machine language code such as code generated by a compiler, as well as high-level language code that may be executed by a computer using an interpreter or the like. The hardware device described above may be configured to operate as one or more software modules to perform the operations of the embodiments, and vice versa.

According to a method and an electronic apparatus for providing classification data of an electrocardiogram signal according to one or more embodiments, the time for analyzing an electrocardiogram signal may be reduced by generating labels that classify the electrocardiogram signal based on category values and providing signal waveforms according to the category values.

Although the embodiments have been described by the limited embodiments and the drawings as described above, various modifications and variations are possible to one of ordinary skill in the art from the above description. For example, the described techniques may be performed in a different order than the described method, and/or components of the described systems, structures, devices, circuits, etc. may be combined or combined in a different manner than the described method, or other components. Or even if replaced or substituted by equivalents, an appropriate result can be achieved.

Therefore, other implementations, other embodiments, and equivalents of the claims fall within the scope of the claims to be described later.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A method for providing classification data of an electrocardiogram signal, the method comprising:
loading, by an electronic apparatus, an electrocardiogram signal;
analyzing, by the electronic apparatus, the electrocardiogram signal and generating labels related to the electrocardiogram signal in association with the electrocardiogram signal;
determining, by the electronic apparatus, category values of signal segments of the electrocardiogram signal based on the labels related to the electrocardiogram signal according to standard classification criteria;
displaying, by the electronic apparatus, the labels related to the electrocardiogram signal and the category values of segments of the electrocardiogram signal on an output unit;
in response to a selection input for a first category value from among the category values, generating, by the electronic apparatus, information regarding an electrocardiogram signal related to the first category value; and
displaying information regarding the electrocardiogram signal related to the first category value on the output unit.

2. The method of claim 1, wherein the information regarding the electrocardiogram signal related to the first category value further comprises information regarding a number of signal segments of the first category value or information regarding a representative signal waveform of the first category value.

3. The method of claim 2, wherein the information regarding the representative signal waveform of the first category value further comprises a number of representative signal waveforms or a number of signal segments of each representative signal waveform.

4. The method of claim 1, further comprising displaying signal waveforms of signal segments classified based on representative signal waveforms of the first category value on the output unit in response to a selection input for information regarding the representative signal waveform of the first category value.

5. The method of claim 1, wherein, in response to a correction input for a first signal segment of the first category value, the electronic apparatus is configured to set a category value of the first signal segment to a second category value,
further comprising correcting classification data to classify the first signal segment into a label including the second category value.

6. The method of claim 1, further comprising, generating, by the electronic apparatus, a report file by converting the information regarding the category values of the electrocardiogram signal and representative signal waveforms of the category values into a report format.

7. The method of claim 1, wherein generating of the labels in association with the signal further comprises classifying the electrocardiogram signal according to pre-set time intervals and generating labels for respective signal sections of the pre-set time intervals.

8. The method of claim 1, wherein, generating of the labels in association with the signal further comprises classifying the electrocardiogram signal according to pre-set heart rates and generating labels for respective signal sections of the pre-set heart rates.

9. The bio-signal measurement apparatus of claim 1, wherein, loading the electrocardiogram signal further comprises loading the electrocardiogram signal by receiving an electrocardiogram signal measured in real time from an electrocardiogram measuring device connected through a communicator or receiving an electrocardiogram signal stored in an external device connected through the communicator.

10. The method of claim 1, wherein
the category values are sub-concepts of the labels, and
one label is defined by including one or more category values.

11. The method of claim 1, wherein displaying the information regarding the electrocardiogram signal related to the first category value on the output unit further comprises, after displaying information including a number of representative signal waveforms of the first category value and a number of signal segments of the first category value, based on an additional input, displaying information including the number of signal segments respectively of representative signal waveforms of the first category value.

12. A computer program stored in a computer-readable storage medium to execute the method of claim 1 by using a computer.
